(19) United States
(12) Patent Application Publication
(10) Pub. No.: US 2026/0126464 A1
(43) Pub. Date: May 7, 2026

(54) AUTOSAMPLER

(71) Applicant: Smith-Root, Inc., Vancouver, WA (US)

(72) Inventors: Austen Clouse Thomas, Port Ludlow, WA (US); Jesse Andrew Howard, Vancouver, WA (US)

(21) Appl. No.: 18/979,192

(22) Filed: Dec. 12, 2024

Related U.S. Application Data
(60) Provisional application No. 63/715,847, filed on Nov. 4, 2024.

(51) Int. Cl. G01N 35/10; G01N 33/18; G01N 35/00

(52) U.S. Cl. CPC G01N 35/1016; G01N 33/18; G01N 35/00722; G01N 2035/00851; G01N 2035/1053

(57) ABSTRACT

An automatic fluid sampler includes a chassis having an interior compartment and an external lid, the chassis having a water inlet and a water outlet, an interior housing in the interior compartment having a compartment lid, an upper manifold in the interior compartment connected to the water inlet and having a water outlet, a lower manifold in the interior compartment connected to the water outlet, an array of slots in the interior housing, each slot having an inlet valve, each slot configured to accept a filter positioned between the inlet valve and the outlet valve, a pump to move the water from the water inlet to the water outlet, a controller configured to operate the pump, the water inlet, and the inlet valve of each slot to cause water from the water inlet to flow to the water outlet through the filter for that slot, a power source.

### RELATED APPLICATIONS

This disclosure is a non-provisional of and claims benefit from U.S. Provisional Application No. 63/715,847, titled “AUTO SAMPLER” filed on Nov. 4, 2024, the disclosure of which is incorporated herein by reference in its entirety.

### TECHNICAL FIELD

This disclosure relates to samplers used in detection of environmental DNA (eDNA), more particularly to robotic samplers.

### BACKGROUND

Analysis of environmental DNA (eDNA) samples provides information about species in a particular location, whether native or invasive. This enables management of ecosystems and natural resources based upon data acquired from the samples. However, current methods and systems for acquisitions of samples generally involve manual sample collection. Manual sample collection takes time, as field personnel have to gather each sample individually. Manual collection by several people also increases the likelihood of variations in collection techniques and protocols, as well as possible limitations of how many samples can be collected in a particular period of time.

### BRIEF DESCRIPTION OF THE DRAWINGS

FIG. 1 shows outer views of an embodiment of an autosampler device.

FIG. 2 shows views of the interior for an embodiment of an autosampler device.

FIG. 3 shows a top view of the interior of an embodiment of an autosampler device.

FIG. 4 shows a circuit diagram for control circuitry for an embodiment of an autosampler device.

FIG. 5 shows a plumbing diagram for an embodiment of an autosampler device.

FIG. 6 show embodiments of filters useable in an autosampler device.

### DETAILED DESCRIPTION OF THE EMBODIMENTS

The embodiments here provide an automated sampling system, also referred to as an autosampler, that can provide multiple samples of water acquired from a body of water very close in time. This alleviates issues with low frequency sampling, improves consistency, and makes it easier for non-expert users to gather samples quickly and in a more consistent manner.

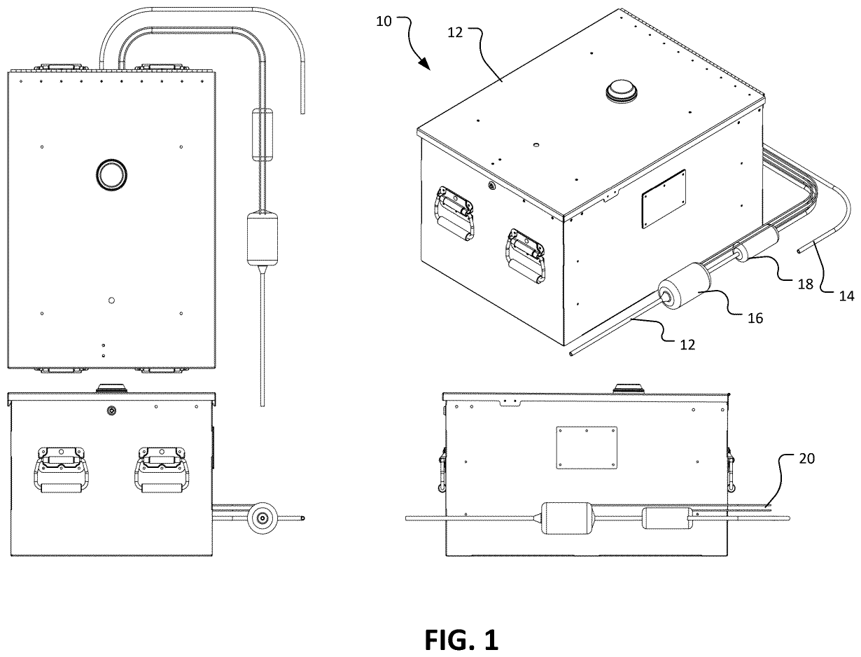

FIG. 1 shows external views of an embodiment of an autosampler 10, with a top view at the upper left, an end view on the lower left, an isometric view on the upper right, and a side view on the lower right. In this embodiment, the autosampler comprises a portable chassis for easy carrying. Users can carry the chassis to whatever area from which they need samples and put the inlet pipes or tubes into the water. The user can position the water inlet tube or pipe 12 to take in water, using a pump 16. The system may include a water level sensor 18 to allow the controller, discussed below, to have data on the water flow that may affect the autosampler operations. The water outlet pipe or tube 14 allows the water to exit the autosampler, such as when the system is purged. The side view allows for a better view of the pump wires 20 that provide power and control to the pump.

One should note that the autosampler of the embodiments here demonstrate one configuration. Variations and modifications to the different components are included in the scope of the claims. The discussion of the specific embodiments is for ease of discussion and understanding, without limitation to any particular implementation.

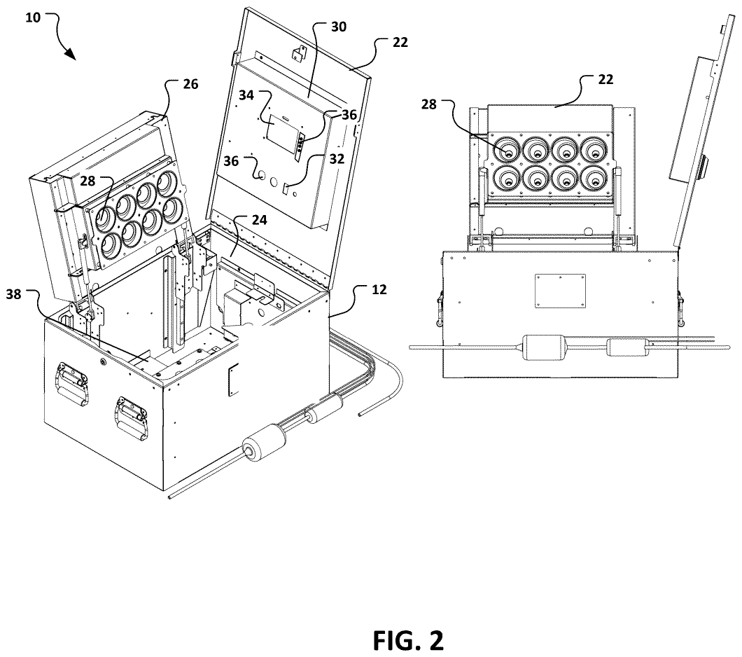

FIG. 2 shows an isometric view of the inside as well as a side view of and embodiment of a chassis that provides a view of the filter slots. With the chassis lid 22 open, one can see the interior compartment 24 that includes an interior housing that has a lid 26, referred to here as the interior housing lid. The compartment led 26 has an array of slots having an upper portion, such as 28 configured to hold water filters. The upper portion of the slot connects to the top of the water filter when inserted, with the bottom portion shown in later views. The embodiment here shows an array of eight slots, with the understanding that other embodiments may employ more or fewer slots. In addition, while this embodiment includes eight slots, the system may operate with fewer than the eight slots provided.

The chassis lid contains a control module 30. The control module 30 may be located elsewhere, but in this embodiment, it resides on the underside of the chassis lid 22. The discussion below will address the components that may reside in the control module. From this view, one can see that the control module has external connections such as 32, a display screen 34, and user controls 36. User controls 36 may include controls adjacent to display 34, as well as control buttons below by the connection, such as a power button, etc. Power may be provided to the controller, the pump, and the valves discussed below, from a battery, such as 38. While the on-board power allows for the ease of moving and locating the autosampler, the use of external power lies within the scope of the claims.

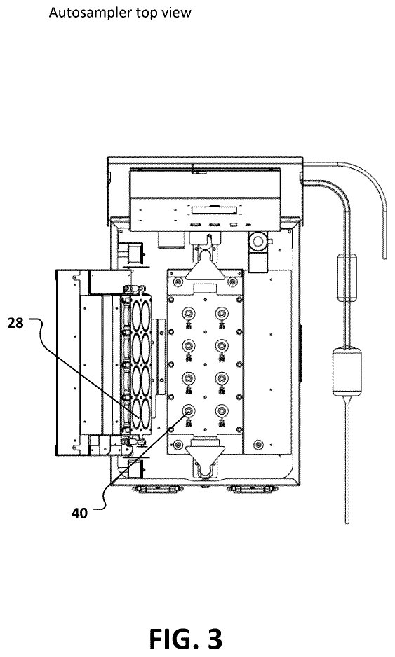

FIG. 3 shows a top view of the interior housing. One can see the bottom portions of the slots such as 40 that correspond to the top portions such as 28. The slot is configured to hold an individual water filter, each of which can receive water from the water inlet under control of the controller as it controls valves. The controller can schedule each filter individually, such as at a particular time for each filter.

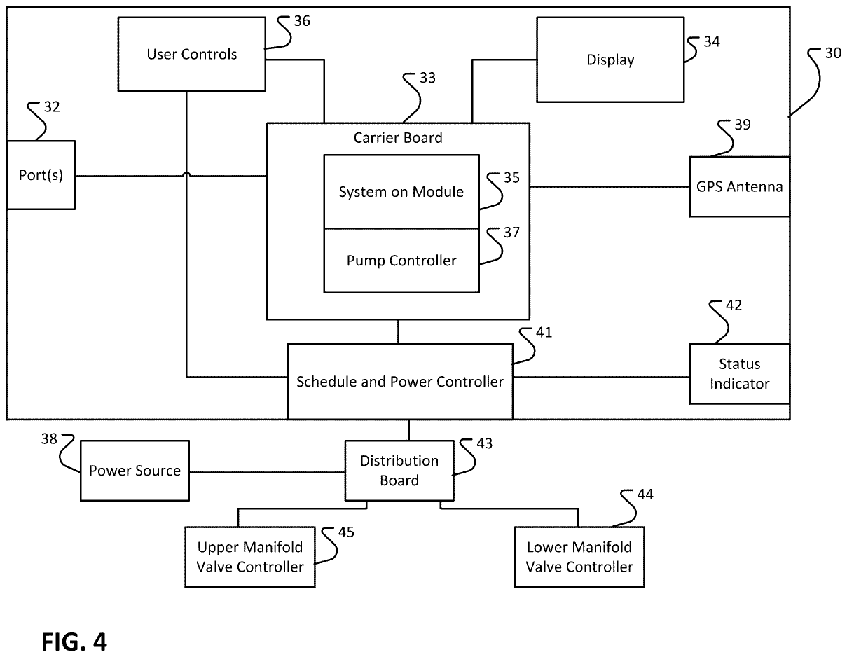

FIG. 4 shows an embodiment of an electrical diagram for module 30. The system on module 35 and the pump controller 37 reside on the carrier board 33. The carrier board is the system board that mounts the system on module and pump controller and provides additional functions and interconnects. The system on module 35 may connect to one or more port(s) 32, such as USB port that allows for downloading of data from the system, as well as uploading system updates.

The user controls 36, shown in FIG. 2, allow the user to control and configure the system software. These controls may include, but are not limited to, a wake/sleep button, which allows a user to wake the unit from sleep, or low power mode, to access controls, and to put the unit back in sleep mode. The wake/sleep button is electrically connected to the schedule and power controller 41. Another control comprises a power switch to allow the user to control unit power. A beeper may be included to provide for audible indication of the sampling progress, typically wired to the schedule and power controller 41. When the unit is off, the battery is disconnected, and no samples are collected. The power switch generally lies between the distribution board 43 and the schedule and power controller 41.

The distribution board 43 will typically reside in the lower housing and provides service disconnect and power distribution. The upper manifold valve controller 45 and the lower manifold valve controller 44 control the manifold valves of their respective manifolds. These are electrically connected to the schedule and power controller 41 through the distribution board 43. The distribution board 43 is connected to battery 38.

The unit may include additional components such as a GPS antenna to allow the unit to set accurate time for samples scheduling. This will typically be connected to the carrier board 33. A status indicator 42 provides indications of the status of the unit and will typically be electrically connected to the schedule and power controller.

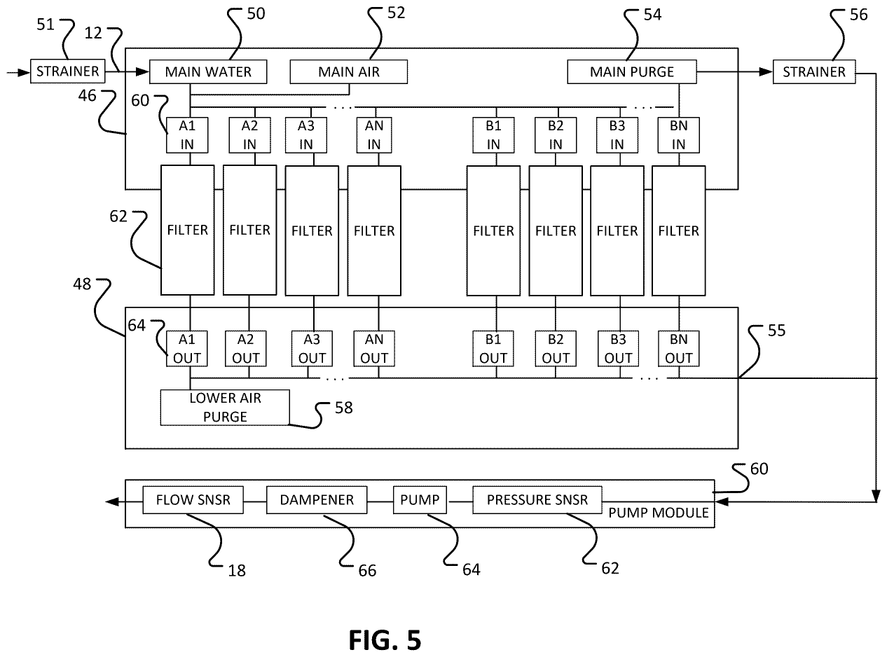

FIG. 5 shows a plumbing diagram for one embodiment of the water flow system of the autosampler. This embodiment employs eight filters, but as mentioned before, may contain more or fewer filters. The upper manifold 46 receives water from the water inlet 12, through inlet strainer 51. The upper manifold routes the water to the various filters, such as 62, when the valves, such as 60, are opened. The lower manifold 48 contains the outlet valves such as 64 for each filter slot.

The overall system valves include a main water valve 50 that controls the flow of water from the inlet to the upper manifold, and a main air valve that lets in air to purge the system. When the system is being purged, the water exits the upper manifold through the main purge valve 54 as an upper manifold outlet. The lower manifold has a lower manifold air purge valve 58. The upper purge valve and water flow path includes a strainer 56. The upper manifold outlet from valve 54 outlets the lower manifold outlet 55 from the valves and the purge path connect to the pump, which may be contained in a pump module. The water flow sensor 18 may reside before the pump, as in FIG. 1, or after the pump as shown in FIG. 5. The pump module may also contain a pressure sensor 62, and a pump dampener 66.

In operation, the system generally undergoes a system flush prior to any filtering. The pump pumps water through the water inlet and purged out of the water outlet. This process does not involve filters. This removes any residual water from any previous use. Once the purge is completed, the controller opens the inlet valves and the outlet valves for an individual filter. In FIG. 5, the controller may open valves 60 and 64 for filter 62, as an example. The water moves through the filter membrane, discussed below, until the water reaches a target volume, or the water flow sensor detects a minimum flow rate. After however many of the filters have collected samples, in some cases only one, the air upper and lower air valves are opened for a limited period of time while the pump runs. This dries the filter membrane and removes any water from the filter and the manifold.

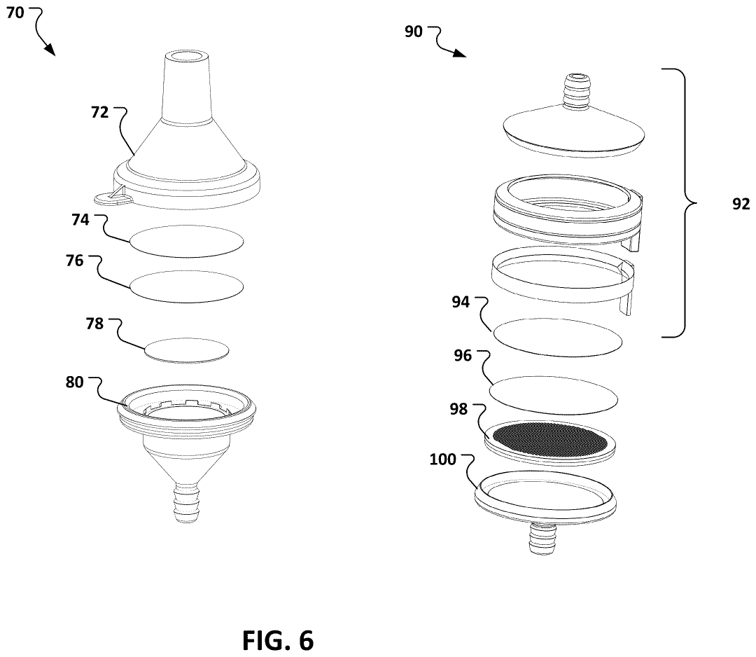

The filter involved in filtering, or sampling, may comprise many different types of filters. In some embodiments, the filter may be “self-desiccating” meaning that it contains a desiccating element of some kind that removes any water left after drying. FIG. 6 shows embodiments of self-desiccating filters.

In a first embodiment of a self-desiccating filter, the filter has a desiccating bottom.

The filter 70 comprises a top 72, a filter membrane 74, a filter backer 76, a steel mesh screen 78, and a desiccating bottom 80. The desiccating bottom comprises a material that removes any water left in the filter membrane after drying. Further discussion of this type of filter can be found in U.S. Pat. No. 12,145,107, issued Nov. 19, 2024, and U.S. Pat. No. 11,471,837, issued Oct. 18, 2022.

In another embodiment, the self-desiccating filter has an internal desiccating element. Filter 90 has a top 92, the example here comprises one of many different configurations, filter membrane 94, filter backer 96 and a desiccating screen 98 that combines the mesh screen with the desiccating element, and a bottom 100. In this type of filter, the bottom does not comprise a desiccant. Further discussion of this type of filter can be found in U.S. patent application Ser. No. 18/904,010, filed Oct. 1, 2024.

These two filters merely provide examples of possible filters that the autosampler can employ. Other types of filters may be employed as well.

Additionally, this written description makes reference to particular features. It is to be understood that the disclosure in this specification includes all possible combinations of those particular features. For example, where a particular feature is disclosed in the context of a particular aspect, that feature can also be used, to the extent possible, in the context of other aspects.

Also, when reference is made in this application to a method having two or more defined steps or operations, the defined steps or operations can be carried out in any order or simultaneously, unless the context excludes those possibilities.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

Although specific aspects of this disclosure have been illustrated and described for purposes of illustration, it will be understood that various modifications may be made without departing from the spirit and scope of the invention. Accordingly, the invention should not be limited except as by the appended claims.

1. An automatic fluid sampler, comprising:
a chassis having an interior compartment and an external lid, the chassis having a water inlet and a water outlet;
an interior housing in the interior compartment, the interior housing having a compartment lid;
an upper manifold in the interior compartment connected to the water inlet, the upper manifold having an upper manifold outlet;
a lower manifold in the interior compartment connected to the water outlet by a lower manifold outlet;
an array of slots in the interior housing, each slot having an inlet valve connected to the upper manifold and an outlet valve connected to the lower manifold, each slot configured to accept a filter positioned between the inlet valve and the outlet valve;
a pump to move the water from the water inlet to the water outlet, the pump having a pump water inlet connected to the upper manifold outlet and lower manifold outlet;
a controller configured to operate the pump, the water inlet, and the inlet valve of each slot to cause water from the water inlet to flow to the water outlet through the filter for that slot; and
a power source to provide power to the pump and the controller.

2. The automatic fluid sampler as claimed in claim 1, further comprising a main water valve and a main water purge valve connected to the upper manifold.

3. The automatic fluid samples as claimed in claim 1, further comprising a strainer connected to the main water purge valve.

4. The automatic fluid sampler as claimed in claim 1, further comprising a main air valve connected to the upper manifold.

5. The automatic fluid sampler as claimed in claim 1, further comprising an air purge valve connected to the lower manifold.

6. The automatic fluid sampler as claimed in claim 1, wherein the pump is contained in a pump module.

7. The automatic fluid sampler as claimed in claim 6, the pump module further comprising a pressure sensor connected to the pump water inlet.

8. The automatic fluid sampler as claimed in claim 6, the pump module further comprising a pump dampener.

9. The automatic fluid sampler as claimed in claim 6, the pump module further comprising a flow sensor between the pump and the water outlet.

10. The automatic fluid sampler as claimed in claim 1, wherein each slot in configured to accept a self-preserving water filter.

11. The automatic fluid sampler as claimed in claim 10, wherein the self-preserving water filter comprises a self-preserving water filter having a desiccating bottom.

12. The automatic fluid sampler as claimed in claim 10, wherein the self-preserving water filter comprises a self-preserving water filter having an internal desiccating element.

13. The automatic fluid sampler as claimed in claim 1, wherein the controller is contained in a control module.

14. The automatic fluid sampler as claimed in claim 13, wherein the control module includes at least one communication port to allow the controller to receive updated executable code.

15. The automatic fluid sampler as claimed in claim 13, wherein the controller includes one or more user controls to allow the user to input schedules and operating parameters for the autosampler.

16. An automatic fluid sampler, comprising:
a chassis having an interior compartment and an external lid, the chassis having a water inlet and a water outlet;
an interior housing in the interior compartment, the interior housing having a compartment lid;
an upper manifold in the interior compartment connected to the water inlet, the upper manifold having an upper manifold outlet;
a lower manifold in the interior compartment connected to the water outlet by a lower manifold outlet;
an array of slots in the interior housing, each slot having an inlet valve connected to the upper manifold and an outlet valve connected to the lower manifold;
at least one self-preserving filter in one of the array of slots positioned between the inlet valve and the outlet valve for the one of the array of slots;
a pump to move the water from the water inlet to the water outlet, the pump having a pump water inlet connected to the upper manifold outlet and lower manifold outlet;
a controller configured to operate the pump, the water inlet, and the inlet valve of each slot to cause water from the water inlet to flow to the water outlet through the filter for that slot; and
a power source to provide power to the pump and the controller.

17. The automatic fluid sampler as claimed in claim 16, wherein the at least one self-preserving water filter comprises a self-preserving water filter having a desiccating bottom.

18. The automatic fluid sampler as claimed in claim 16, wherein the at least one self-preserving water filter comprises a self-preserving water filter having an internal desiccating element.

19. An automatic fluid sampler, comprising:
a chassis having an interior compartment and an external lid, the chassis having a water inlet and a water outlet;
an interior housing in the interior compartment, the interior housing having a compartment lid;
an upper manifold in the interior compartment connected to the water inlet, the upper manifold having an upper manifold outlet;
a lower manifold in the interior compartment connected to the water outlet by a lower manifold outlet;
an array of eight slots in the interior housing, each slot having an inlet valve connected to the upper manifold and an outlet valve connected to the lower manifold;
a self-preserving filter in each of the array of eight slots positioned between the inlet valve and the outlet valve for the one of the array of slots;
a pump to move the water from the water inlet to the water outlet, the pump having a pump water inlet connected to the upper manifold outlet and lower manifold outlet;
user controls to allow the user to provide inputs to the system;
a display;
a system on module controller mounted on the external lid, the system on module controller configured to accept user control signals, store data in a memory, and display information on the display;
a distribution board;
a pump controller to control operation of the pump, monitor sensors near the water outlet, and send control signals to the distribution board, the distribution board to send power signals to the inlet valves and the outlet valves; and
a power source to provide power to the pump and the controller.
